(19) United States
(12) Patent Application Publication
(10) Pub. No.: US 2016/0129059 A1
(43) Pub. Date: May 12, 2016

(54) MICROORGANISM BIOMASS FOR PREVENTION AND REDUCTION OF THE ADVERSE EFFECTS OF MYCOTOXINS IN DIGESTIVE TRACT

(71) Applicant: NESTE OIL CORPORATION, Espoo (FI)

(72) Inventors: Jukka-Pekka PASANEN, Helsinki (FI); Juha APAJALAHTI, Helsinki (FI); Eero PENNALA, Espoo (FI)

(21) Appl. No.: 14/893,778

(22) PCT Filed: May 26, 2014
(86) PCT No.: PCT/EP2014/060861

§ 371 Date: Nov. 24, 2015

(30) Foreign Application Priority Data
May 24, 2013 (EP) 13169253.5

(51) Int. Cl. A61K 36/062; C12N 1/14; A61K 36/06

(52) U.S. Cl. CPC A61K 36/062; A61K 36/06; C12N 1/14; A23L 1/3018; A23V 2002/00

(57) ABSTRACT

The present invention relates to microorganism biomass and a feed or food composition which can be used in preventing or reducing the adverse effects of mycotoxins in animal or human digestive tract. The biomass comprises non-living microorganism biomass obtainable by cultivating microorganism strains on a cultivation medium comprising lignocellulosic material.

### FIELD OF THE INVENTION

The present invention relates to microorganism biomass or a feed or food composition comprising said biomass for use in preventing or reducing the adverse effects of mycotoxins in animal or human digestive tract. The invention relates also to a feed or food composition and to a method for producing it. The present invention relates also to a method for improving the well-being and increasing the productivity of an animal.

### BACKGROUND OF THE INVENTION

Mycotoxins often contaminate feedstuffs. Mycotoxins have multiple toxic effects which cause reduced feed intake and impaired animal performance. Mycotoxins are toxic metabolites produced by molds and fungi and can be present in feed raw materials already at harvest or produced during suboptimal storage. Fungal spores are found everywhere in the environment and therefore mycotoxin contamination in feeds is a major concern to farm animal industry. Mycotoxins can induce acute and long-term chronic diseases on animals and, for example, reduced body weight gain and fertility leading to economic losses. In addition, mycotoxins increase susceptibility to viral and bacterial diseases. Besides the fact that mycotoxins decrease animal performance, toxin residues in milk, meat and eggs pose a threat to human end consumers. Mycotoxins are stable and bioaccumulating molecules, and therefore, very difficult to eliminate from contaminated food (Boudergue et al. 2009. Review of mycotoxin-detoxifying agents used as feed additives: mode of action, efficacy and feed/food safety; CFP/EFSA/FEEDAP/2009/01)

US 2012/0027747 (Tranquil et al.) describes a composition for adsorbing mycotoxins comprising biomass of one or more filamentous fungi. It is suggested that the biomass is produced either as a by-product of fungal fermentation or in a dedicated process.

The binding of mycotoxins has been reported by using different adsorbents, for example activated charcoal and aluminosilicates. In addition, yeasts grown on saccharides and yeast products (yeast cell wall extracts) have been used in mycotoxin binding. Commercial yeast polysaccharides including e.g MTB100®, Alltech Inc.) are reported to adsorb mycotoxins.

Although some attempts to eliminate or reduce the effects of mycotoxins in digestive tract are known from the prior art, there is still a need for efficient products and methods which could be used to prevent or reduce the adverse effects of mycotoxins in digestive tract in animals and human.

### SUMMARY OF THE INVENTION

It is one object of the present invention to provide a solution to problems encountered in the prior art. Specifically, the present invention aims to provide a solution to problems encountered in the well-being of animals and human. Furthermore, the present invention aims to increase the productivity of animals.

In particular, it is one object of the present invention to provide a solution, which enables prevention or reduction of the adverse effects of mycotoxins in animal or human digestive tract.

To achieve these objects the invention is characterized by the features that are enlisted in the independent claims. Other claims represent the preferred embodiments of the invention.

The invention is based on the finding that microorganism biomass prevents and reduces the adverse effects of mycotoxins in animal or human digestive tract.

It has now been surprisingly found that microorganism biomass, in particular biomass, which has been obtained by cultivating microorganisms on a cultivation medium comprising lignocellulosic material has this ability of preventing and reducing the adverse effects of mycotoxins.

Hence, in one aspect, the present invention provides microorganism biomass for use in preventing or reducing the adverse effects of mycotoxins in animal or human digestive tract as defined in claim 1.

In another aspect, the present invention provides a feed or food composition for use in preventing or reducing the adverse effects of mycotoxins in animal or human digestive tract as defined in claim 9.

In a further aspect, the present invention provides a feed or food composition as defined in claim 14.

In a still further aspect, the present invention provides a method for improving the well-being and increasing the productivity of an animal as defined in claim 16.

In a still further aspect, the present invention provides a method for producing a feed or food composition.

In still further aspects, the present invention provides a method, a microorganism biomass, and a feed or food composition for preventing or reducing the adverse effects of mycotoxins in animal or human digestive tract.

### BRIEF DESCRIPTION OF THE FIGURES

FIGS. 1 and 5 show the binding of microorganism biomass of aflatoxin.

FIGS. 2 and 6 show the binding of microorganism biomass of ochratoxin.

FIGS. 3 and 7 show the binding of microorganism biomass of zearaleone.

FIGS. 4 and 8 show the binding of microorganism biomass of vomitoxin.

FIG. 9 shows the binding of microorganism biomass of Fumonisin B1.

FIG. 10 shows the binding of microorganism biomass of T2 toxin.

### DETAILED DESCRIPTION OF THE INVENTION

### Definitions

The present invention provides microorganism biomass for use in preventing or reducing the adverse effects of mycotoxins in animal or human digestive tract. Such biomass is obtainable by cultivating said microorganisms on a cultivation medium comprising lignocellulosic material.

According to this disclosure microorganism biomass obtainable by cultivating microorganism strains in a cultivation medium comprising lignocellulosic material, typically lignocellulose hydrolysate or saccharides has been shown to reduce the bioavailability of mycotoxins originating from contaminated feed.

As disclosed herein the microorganism biomass grown on lignocellulosic material, in particular lignocellulosic hydrolysate, is able to bind a wide range of mycotoxins. In addition, said biomass is able to bind mycotoxins both at the pH of the acidic stomach and neutral small intestine and colon.

The binding effect of adsorbing agents is based on that said agents physically bind mycotoxins and inhibit their uptake from the digestive tract of animals and the subsequent distribution of the blood and target organs. The toxin-adsorbing agent complex passes through the animal digestive tract and is voided via the faeces, thus minimizing the exposure of animal tissues to mycotoxins.

By “lignocellulosic material” is meant any material which comprises lignocellulose. Lignocellulosic material comprises lignocellulose, fragments of lignocellulose or hydrolysis products of lignocellulose including saccharides, such as mono-, di-, and/or oligosaccharides originating from lignocellulose. “Lignocellulosic material” may comprise also other components in addition to lignocellulose such as other components from wood or herbaceous plants.

“Lignocellulose” comprises carbohydrate polymers cellulose and hemicellulose and aromatic polymer lignin. Lignocellulosic material include but is not limited to woody plants or non-woody, herbaceous plants or other materials containing cellulose and/or hemicellulose: Materials can be agricultural residues (such as wheat straw, rice straw, chaff, hulls, corn stover, sugarcane, bagasse), dedicated energy crops (such as switchgrass, Miscanthus, reed canary grass, willow, water hyacinth), wood materials or residues (including sawmill and pulp and/or paper mill residues or fractions, such as hemicellulose, spent sulphite liquor, waste fibre and/or primary sludge), moss or peat, or municipal paper waste. The term lignocellulosic material comprises also low lignin materials, materials such as macroalgae biomass. In addition, the materials comprise also hemicellulose or cellulose fractions from industrial practices. The term lignocellulosic material encompasses any kind of cellulose fraction. The raw materials or certain fractions, such as hemicellulose and/or cellulose, of raw materials from different origin, plant species, or industrial processes can be mixed together and used as raw materials for cultivating microorganism biomass according to this disclosure.

The term lignocellulosic material comprises at least 50 wt % lignocellulose, preferably at least 60 wt % lignocellulose, more preferably at least 70 wt % lignocellulose, most preferably at least 80 wt % lignocellulose. Usually lignocellulosic material comprises 60-95 wt % lignocellulose, typically 70-90 wt %, or 80-90 wt % lignocellulose.

“A cultivation medium comprising lignocellulosic material” means a cultivation medium for cultivating a microorganism, which medium comprises lignocellulose, fragments of lignocellulose or hydrolysis products of lignocellulose including nitrogen compounds originating from proteins and metals, and other components necessary for cultivating said microorganism, such as a source of nitrogen, phosphorus, inorganic salts and/or trace elements. Lignocellulose may function as a carbon source for said microorganism, but it may also have other functions in the cultivation medium.

“Hydrolysis” refers here to saccharification of polymeric sugars to sugar oligomers and monomers. Saccharification is typically carried out in two phases: first the substrate i.e. lignocellulosic material or lignocellulose is hydrolyzed by thermochemical or chemical methods and then by using enzymes capable of hydrolysing polymeric sugars. Alternatively and depending on the lignocellulosic material saccharification can be carried out by using thermochemical or chemical methods or by enzymes capable of hydrolysing polymeric sugars or some combination of these methods. Chemical methods include, but are not limited to acid treatment.

In some embodiments of the invention, the microorganism cultivated on a medium comprising lignocellulosic material is able to hydrolyse lignocellulose to sugar oligomers and monomers. In other preferred embodiments lignocellulosic material or feedstock comprising polymeric sugars is hydrolyzed to monomeric sugars thermochemically and/or chemically and/or by enzymes before cultivation. In preferred embodiments of the invention, lignocellulosic material containing polymeric sugars is hydrolysed thermochemically and/or chemically and/or by enzymes to contain oligomeric sugars and the microorganism cultivated on a medium comprising lignocellulose is able to utilize these sugar oligomers.

By “lignocellulose hydrolysate” is meant the hydrolysis products of lignocellulose or lignocellulosic material comprising cellulose and/or hemicellulose, oligosaccharides, mono- and/or disaccharides, acetic acid, formic acid, other organic acids, furfural, hydroxymethyl furfural, levulinic acid, phenolic compounds, other hydrolysis and/or degradation products formed from lignin, cellulose, hemicellulose and/or other components of lignocellulose, nitrogen compounds originating from proteins, metals and/or non-hydrolyzed or partly hydrolyzed fragments of lignocellulose.

According to this disclosure the treatment of lignocellulose and production of lignocellulose hydrolysate for cultivation of microbial biomass can be done with any method known in the art or developed in the future. Methods include but are not limited to thermochemical treatment, steam explosion, hot water extraction, autohydrolysis, sub critical water treatment, super critical water treatment, strong acid treatment, mild acid treatment, alkaline treatment (e.g. lime, ammonia), Organosolv treatment (e.g. alcohols, organic acids), mechanical treatment, thermomechanical treatment and ionic liquid treatment. These treatments methods can be combined with enzymatic treatment.

“A cultivation medium comprising saccharides” means here a cultivation medium, which comprises mono-, di-, and/or oligosaccharides from lignocellulose.

“A cultivation medium comprising pure saccharides” means here a cultivation medium, which comprises mono-, di- and/or oligosaccharides, which are not produced by hydrolysis of lignocellulose. Pure saccharides include, e.g. starch or starch derived sugars, sugar cane or sugar beet derived sugars.

By “a microorganism” is meant any microorganism, typically a fungus, preferably a filamentous fungus or yeast, a heterotrophic algae, a bacterium or an archaebacterium, which microorganism is capable of producing cellular biomass.

Microorganism is able to produce microbial biomass when grown on cultivation medium comprising lignocellulosic material. Preferably said microorganism is a filamentous fungus or yeast. More preferably the microorganism biomass is fungal biomass, still more preferably obtainable from cultivation of filamentous fungi or yeasts, most preferably filamentous fungi from genus Aspergillus and/or Mortierella or yeasts from genus Rhodosporidium and/or Lipomyces.

“Cell mass” or “Microbial biomass” or “microorganism biomass” are used here synonymously and stand for a solid, semi-solid or flowing material fraction, which contains microorganisms or is treated for the recovery of specific products produced by said microorganism. Microorganism biomass comprises microorganism cells or residues of microorganism cells.

Typically microorganism biomass according to this disclosure means biomass of non-living microorganisms. Methods used for the recovery of co-products and/or microorganism biomass typically comprise treatments which destroy the structure of the microorganism cell, such as disrupt microbial cell wall. Microorganism biomass thus means in particular non-living microorganisms or their residues. According to this disclosure a microorganism can be cultivated for biomass production or it can be cultivated under conditions that permit the microorganism to produce desired products, such as lipids, ethanol, enzymes, or other economically valuable co-products. Preferably the microorganism biomass according to this disclosure is obtained after recovery of lipids from microbial biomass.

Preferably, by “a microorganism” is meant here a lipid-producing, in another word oleaginous, microorganism. When a lipid-producing microorganism has been used for single cell oil production, the microorganism biomass is residual biomass from a single cell oil production process.

By “lipid producing microorganism” or “oleaginous microorganism” is meant a microorganism that is able to produce and accumulate in their cell biomass more than 15% lipids from their dry cell biomass weight when cultivated in suitable conditions for lipid production. Alternatively or in addition microorganism may be able to excrete lipids outside the cells e.g. to cultivation medium.

Single-cell oil stands typically for an intracellular lipid that has been intracellularly synthesized by a microorganism, lipids excreted by the cell, as well as lipids present in the structural parts of a cell, such as in membrane systems.

By “single-cell oil production process” is meant a process where microorganisms are used to produce oils. In the process, microorganisms are cultivated on organic carbon sources and microorganisms are let to produce oil. Microorganisms can store the oil intracellularly or excrete it out from the cell. Organic carbon source can be lignocellulosic material. Single-cell oil process typically utilizes microorganisms, such as oleaginous microorganisms, that are capable of producing lipids efficiently.

“Lipid recovery” or “oil recovery” refers to a process, in which the lipid (intracellular lipid) is recovered by mechanical, chemical, biochemical, thermomechanical or autocatalytic methods or by a combination of these methods from the microorganism cells.

After cultivation of microorganisms for production of oil, microorganisms containing lipids may be separated from culture medium by any known methods, such as by using a filtration or decanting techniques. Alternatively, centrifugation with industrial scale commercial centrifuges of large volume capacity may be used to separate the desired products.

In various embodiments of the invention, oil, or precursors for oil, may be recovered from cell biomass or culture broth using any method known in the art or developed in the future. Such methods, include, but are not limited to extraction with organic solvents or mechanical pressing. In various embodiments of the invention, microorganism cells may be disrupted to facilitate the separation of oil and other components. Any method known for cell disruption may be used, such as ultrasonication, osmotic shock, mechanical shear force, cold press, thermal shock, enzyme-catalyzed or self-directed autolysis.

“Residual cell mass” or “residual microbial biomass” or “residual microorganism biomass” are used here synonymously and stand for a solid, semi-solid or flowing material fraction, which contains microorganisms treated for the recovery of intracellular lipids and/or other products.

The term “lipid” refers to a fatty substance, whose molecule generally contains, as a part, an aliphatic hydrocarbon chain, which dissolves in nonpolar organic solvents but is poorly soluble in water. Lipids are an essential group of large molecules in living cells. Lipids are, for example, fats, oils, waxes, wax esters, sterols, terpenoids, isoprenoids, carotenoids, polyhydroxyalkanoates, fatty acids, fatty alcohols, fatty acid esters, phospholipids, glycolipids, sphingolipids and acylglycerols. The term “lipid” and “oil” are used in this description synonymously. The term “acylglycerol” refers to an ester of glycerol and fatty acids. Acylglycerols occur naturally as fats and fatty oils. Examples of acylglycerols include triacylglycerols (TAGs, triglycerides), diacylglycerols (diglycerides) and monoacylglycerols (monoglycerides).

In embodiments where microorganism biomass is cultivated on lignocellulosic material, in particular lignocellulose hydrolysate, it may comprise also hydrolysis products or residues of lignocellulose. Such residues are for example, acetic acid, formic acid, other organic acids, furfural, hydroxymethyl furfural, levulinic acid, phenolic compounds, other hydrolysis or degradation products formed from lignin, cellulose or hemicellulose or other components of lignocellulose, or non-hydrolyzed or partly hydrolyzed fragments of lignocellulose. Hence, if microorganism biomass is cultivated on lignocellulose hydrolysate, it typically comprises lignocellulose hydrolysis products or residues, which the microorganism has not utilized during cultivation or which the microorganism has not been able to utilize, for example lignin, polysaccharides and mono-, di- or oligosaccharides. The amount of lignocellulose hydrolysis products or residues is typically 0.01-20 wt % (dry weight), usually 0.5-10 wt % of dry weight of the microorganism biomass. The amount of phenolic compounds originating from lignocellulosic materials is typically 0.01-10 wt % (dry weight), usually 0.05-5 wt % of dry weight of the microorganism biomass. Depending on the source of the lignocellulosic material and on other components of the cultivation medium, “microorganism biomass” may comprise for example residues of various proteins and lipids.

In preferred embodiments of the invention, the microorganism is cultivated on lignocellulosic hydrolysate containing lignocellulose hydrolysis or degradation products not utilized by microorganism, such as phenolic compounds and furfural. These compounds are formed in the hydrolysis of lignocellulose. The lignocellulose hydrolysis can be performed by thermochemical treatment, steam explosion, hot water extraction, autohydrolysis, sub critical water treatment, super critical water treatment, strong acid treatment, mild acid treatment, alkaline treatment (e.g. lime, ammonia), Organosolv treatment (e.g. alcohols, organic acids), mechanical treatment, thermomechanical treatment, ionic liquid treatment in addition to enzymatic treatment.

Without being bound by any theory it seems that lignocellulosic residues in microorganism biomass may be at least partly responsible for the prevention or reduction of adverse effect of mycotoxins in digestive tract. The components of hydrolysate, such as phenolic compounds and metals may adsorb to the cell wall of microorganisms and be carried on with the microbial biomass. Alternatively the microbial cell walls may be modified by the components of lignocellulose hydrolysate.

The capability of microorganism biomass according to this disclosure to prevent or reduce the adverse effect of mycotoxins may also be due to process driven changes in rheology of the biomass and/or changes in the cell wall composition of the biomass. These changes in biomass rheology and composition of the cell wall can potentially be due to single cell production process and/or due to the use of lignocellulosic hydrolysates in the cultivation.

Single cell oil production process typically uses nutrient starvation, such as nitrogen or phosphorus starvation to allow microorganisms to produce and accumulate oil. The starvation in single cell production process can result in changes in biomass rheology and composition of the cell wall.

Microorganism biomass grown on lignocellulosic material, in particular lignocellulose hydrolysate binds according to this disclosure a wide range of mycotoxins including, but not limited to aflatoxin, ochratoxin, zearaleon, vomitoxin, fumonisin and T2 toxin. This encompasses thus also vomitoxin, which is not bound for example by commercial mycotoxin binder hydrated sodium calcium aluminosilicate (HSCAS). Microorganism biomass grown on lignocellulosic material, in particular lignocellulose hydrolysate binds also mycotoxins at wide pH range, and is therefore able to bind mycotoxins in various pHs of the digestive tract, in stomach (pH 2.5) and in small intestinal conditions (pH 6.5). Furthermore, the microorganism biomass grown in particular on lignocellulose hydrolysate is a more effective mycotoxin binder than the microorganism biomass grown on pure saccharides. However, this effect depends also on the mycotoxin.

Preferred microorganism strains for the purposes of the present invention are from the species and genera listed below:

Preferred fungal strains are from species from genera Aspergillus such as Asrgillus oryzae, Mortierella such as Mortierella isabellina, Chaetomium, Claviceps, Cladosporidium, Cunninghamella, Emericella, Fusarium, Glomus, Mucor, Paecilomyces, Penicillium, Pseudozyma, Pythium, Rhizopus, Tremella, Trichoderma, Zygorhynchus, Humicola, Cladosporium, Malbranchea, Umbelopsis such as Umbelopsis isabellina and Ustilago. Most preferred fungal species are from genera Aspergillus and/or Mortierella. Preferred fungi are those fungi capable of producing effectively lipids.

Preferred yeast strains are those belonging to species from genera Clavispora, Geotrichum, Deparyomyces, Pachysolen, Kluyveromyces, Galactomyces, Hansenula, Leucosporidium, Saccharomyces, Sporobolomyces, Sporidiobolus, Waltomyces, Endomycopsis, Cryptococcus, such as Cryptococcus curvatus, Rhodosporidium, such as Rohodosporidium toruloides, Rhodotorula, such as Rhodotorula glutinis, Yarrowia, such as Yarrowia lipolytica, Pichia, such as Pichia stipitis, Candida such as Candida curvata, Lipomyces such as Lipomyces starkeyi and Trichosporon such as Trichosporon cutaneum or Trichosporon pullulans. Preferred yeasts are those yeasts capable of producing effectively lipids.

Preferred bacteria are those belonging to the species from genera Acinetobacter, Actinobacter, Alcanivorax, Aerogenes, Anabaena, Arthrobacter, Bacillus, Clostridium, Dietzia, Gordonia, Escherichia, Flexibacterium, Micrococcus, Mycobacterium, Nocardia, Nostoc, Oscillatoria, Pseudomonas, Rhodococcus, Rhodomicrobium, Rhodopseudomonas, Shewanella, Shigella, Streptomyces and Vibrio. Preferred bacteria are those bacteria capable of producing effectively lipids.

Most preferred algae are microalgae, such as microalgae species from genera comprising Achnantes, Amphiprora, Amphora, Ankistrodesmus, Attheya, Boeklovia, Botryococcus, Biddulphia, Brachiomonas, Bracteococcus, Carteria, Chaetoceros, Characium, Chlamydomonas, Crypthecodinium, Cryptomonas, Chlorella, Chlorococcum, Chrysophaera, Coccochloris, Cocconeis, Cyclotella, Cylindrotheca, Dunaliella, Ellipsoidon, Entomoneis, Euglena, Eremosphaera, Extubocellulus, Franceia, Fragilaria, Gleothamnion, Hantzschia, Haematococcus, Hormotilopsis, Hymenomonas, lsochrysis, Lepocinclis, Melosira, Minidiscus, Micractinum, Monallanthus, Monoraphidium, Muriellopsis, Nannochloris, Nannochloropsis, Navicula, Neochloris, Nephroselmis, Nitzschia Ochromonas, Oedogonium, Oocystis, Papiliocellulus, Parachlorella, Pascheria, Pavlova, Peridinium, Phaeodactylum, Plankthothrix, Platymonas, Pleurochrysis, Pleurosigma, Porphyridium, Prototheca, Prymnesium, Pseudochlorella, Pyramimonas, Pyrobotrus, Radiosphaera, Rhodomonas, Rhodosorus, Sarcinoid, Scenedesmus, Schizochytrium, Scrippsiella, Seminavis, Skeletonema, Spirogyra, Stichococcus, Synedra, Tetraedron, Tetraselmis, Thalassiosira, Trachyneis, Traustrochytrium, Trentepholia, Ulkenia, Viridiella, and Volvox. Preferred microalgae are those microalgae capable of growing heterotrophically and producing effectively lipids. The organisms belonging to the genera Schizochytrium, Thraustochytrium and Crypthecodinium and Ulkenia are sometimes called as marine fungi.

According to the present disclosure microorganism biomass obtained by cultivating microorganisms on a cultivation medium comprising lignocellulosic material prevents or reduces the adverse effects of mycotoxins in animal or human digestive tract, preferably in animal or human stomach, small intestine and/or colon.

By “mycotoxin” is here meant any secondary metabolite produced by fungi that cause a toxic response (mycotoxicosis) when ingested by animals or human.

Mycotoxins comprise for example:

Aflatoxin, in particular B1, B2, G1 and G2, for example AFB1. Aflatoxin is synthesized by fungi of Aspergillus genus. It is found in all major cereal crops. It is typically found in high moisture and temperature. It causes liver disease, and carcinogenic and teratogenic effects.

Ochratoxin, in particular Ochratoxin A, is synthesized by fungi of Penicillium and Aspergillus genera. It causes nephrotoxicity, mild liver damage and immune suppression. Especially chickens are susceptible to ochratoxin.

Zearaleone in particular Zearaleone (ZEA) is synthesized by fungi of Fusarium genus. It causes estrogenic effects, atrophy of ovaries and testicles and abortion. It binds estrogen receptors in farm animals and thereby disrupts estrogenic hormone binding. Especially swine are sensitive to zearaleone.

Trichothecenes, in particular Vomitoxin (deoxyvalenol, DON) is synthesized by fungi of Fusarium genus. They cause Immunologic effects, diarrhoea, reduced feed intake and haemorrhages in animals.

Fumonisin, in particular fumonisin B1, B2 and B3 synthesized by fungi of Fusarium genus. It causes pulmonary edema, nephrotoxicity, hepatotoxicity in animals.

By “preventing or reducing the adverse effects of mycotoxins” is meant here the increase in well-being and productivity of animals, the productivity of said animals being decreased by animal diseases or disorders caused by mycotoxins, or high mortality of said animals being caused by mycotoxins. In humans preventing or reducing the adverse effects of mycotoxins means the increase in well-being and better health.

The present invention provides also a feed or food composition for use in preventing or reducing the adverse effects of mycotoxins in animal or human digestive tract. Said feed or food composition comprises the microorganism biomass according to this disclosure.

The use of a feed composition for use in reducing the amount of mycotoxins in feed results also in reduction of mycotoxins in products sold to end consumers, such as milk, eggs, or meat.

The feed or food composition comprises microorganism biomass preferably 0.001-20 wt %, more preferably 0.1-3 wt % dry weight of dry weight of said composition.

In some embodiments of the invention the composition is a ruminant or monogastric animal, such as pigs or poultry feed composition. The composition comprises microorganism biomass and components or ingredients suitable for ruminant or monogastric animal feed composition, such as source of protein and fibre components.

By “feed or food ingredient” is here meant a component part or constituent of any combination or mixture making up a feed or food, whether or not it has a nutritional value in the animal's or human's diet, including feed or food additives. Ingredients are of plant, animal or aquatic origin, or other organic or inorganic substances.

In some other embodiments of the invention the composition is a fish feed composition. The composition comprises microorganism biomass and components or ingredients suitable for fish feed composition, such as source of protein and fat components.

In further embodiments of the invention the food composition is a nutritional product for human use. The composition comprises microorganism biomass and components or ingredients suitable for food composition. Such components or ingredients comprise for example salt and sugar or other flavour giving ingredients.

The microorganism biomass may be used in the feed or food composition as a food or feed topping, food or feed extender or feed or food supplement.

The present invention provides also a feed or food composition, which comprises preferably 0.001-20 wt %, more preferably 0.1-3 wt % dry weight of dry weight of said composition non-living microorganism biomass, said microorganism biomass being obtainable as disclosed herein, and preventing or reducing the adverse effects of mycotoxins in animal or human digestive tract.

The present invention provides also a method for producing a feed or food composition. Preferable the method comprises the steps of
cultivating a microorganism on a cultivation medium comprising lignocellulosic material;
   collecting microorganism cells from said cultivation medium;
   optionally rupturing or lysing the cultured cells;
separating the solid phase containing microbial biomass and/or separating the cell wall from the soluble intracellular components and obtaining a cell wall extract and optionally extracting said biomass or cell extract by organic acid, typically hexane;
drying and optionally desolventizing the separated residual microbial biomass or cell extract; and
adding said cell mass or cell extract to feed or food.

The present invention provides also a method for producing a feed or food composition from single cell oil production process. Preferable the method comprises the steps of
cultivating a microorganism on a cultivation medium comprising lignocellulosic material;
allowing microorganisms to produce oil, preferably under nutrient starvation;
collecting oil-rich microorganism cells from cultivation medium and optionally drying the cells;
rupturing or lysing the cultured cells;
recovering oil from microorganisms cells typically by solvent, e.g. hexane, said extraction creating liquid phase containing oil and residual microorganism biomass,
separating the residual microorganism biomass;
drying the separated residual microorganism biomass; and
adding said microorganism biomass to feed or food.

Separated residual microorganism biomass may be treated mechanically, thermochemically, chemically or enzymatically prior to adding to feed or food.

Advantageously a feed or food composition is prepared by mixing a specific amount of microorganism biomass or cell wall extract to feed or food. A feed or food composition is prepared to comprise preferably 0.001-20 wt %, more preferably 0.1-3 wt % (dry weight) of dry weight of said composition microorganism biomass obtainable as disclosed herein.

Advantageously the microorganism biomass comprises residues of lignocellulose. The amount of lignocellulose hydrolysis products or residues is typically 0.01-20 wt % (dry weight), usually 0.5-10 wt % of dry weight of the microorganism biomass. The amount of phenolic compounds originating from lignocellulosic materials is typically 0.01-10 wt % (dry weight), usually 0.05-5 wt % of dry weight of the microorganism biomass.

The present invention provides also a method for improving the well-being and increasing the productivity of an animal. The method comprises feeding to said animal a feed composition comprising non-living microorganism biomass, said biomass being obtainable by cultivating a microorganism on a cultivation medium comprising lignocellulosic material as disclosed herein. Preferably the composition comprises 0.001-20 wt %, more preferably 0.1-3 wt % dry weight of dry weight of said composition microorganism biomass.

Preferably the microorganism biomass is microorganism biomass as disclosed here earlier.

Advantageously the microorganism biomass comprises residues of lignocellulose. The amount of lignocellulose hydrolysis products or residues is typically 0.01-20 wt % (dry weight), usually 0.5-10 wt % of dry weight of the microorganism biomass. The amount of phenolic compounds originating from lignocellulosic materials is typically 0.01-10 wt % (dry weight), usually 0.05-5 wt % of dry weight of the microorganism biomass.

In some embodiments of the invention the composition is a ruminant or monogastric animal feed composition.

In some other embodiments of the invention the composition is a fish feed composition.

In further embodiments of the invention the food composition is a nutritional product for human use.

The microorganism biomass may be used in the feed or food composition as a feed or food topping, feed or food extender or feed or food supplement.

According to this disclosure the microorganism biomass obtainable by cultivating microorganisms on a medium comprising lignocellulosic material is used to prevent or reduce the adverse effects of mycotoxins in animal or human digestive tract.

The present invention has been exemplified by showing that microorganism biomass, in particular fungal biomass obtainable from cultivation of Aspergillus Mortierella, and Rhodosporidium with pure saccharides or with lignocellulosic hydrolysate can be used as a binding agent for mycotoxins in the animal digestive tract.

The microbial biomasses from microorganisms grown on lignocellulosic material, in particular lignocellulosic hydrolysates inhibit also the binding of other mycotoxins to intestinal epithelium than those described in the Examples and reduce the adverse effects by other mycotoxins than those described in the Examples to animals or humans.

Microorganism biomass originating from cultivation using pure saccharides or lignocellulosic hydrolysate was used to bind mycotoxins. The ability of fungal biomass of genus Aspergillus and Mortierella and of yeast biomass of genus Rhodosporidium to bind aflatoxin, ochratoxin, zearaleone, at pH of 2.5 and 6.5 was studied and compared with hydrated sodium calcium aluminosilicate (HSCAS) as positive control. Studies with fumonisin and T2 toxin were carried out with yeast biomass of genus Rhodosporidium as well. According to the results microorganism biomass, in particular residual microorganism biomass cultivated on a lignocellulosic hydrolysate was found to be more effective binder for all tested mycotoxins than residual fungal or yeast biomass cultivated with pure saccharides. The basis of the observed effect is not precisely known but is probably result of changes of rheology of the biomass and/or changes in the cell wall composition.

The advantage of the present invention is the enhanced ability of the microorganism biomass grown on lignocellulose hydrolysate to bind a wide range of mycotoxins both at the pH of the acidic stomach and neutral small intestine and colon.

In the present invention it was shown that, the microorganism biomass grown on lignocellulosic material can bind various different mycotoxins.

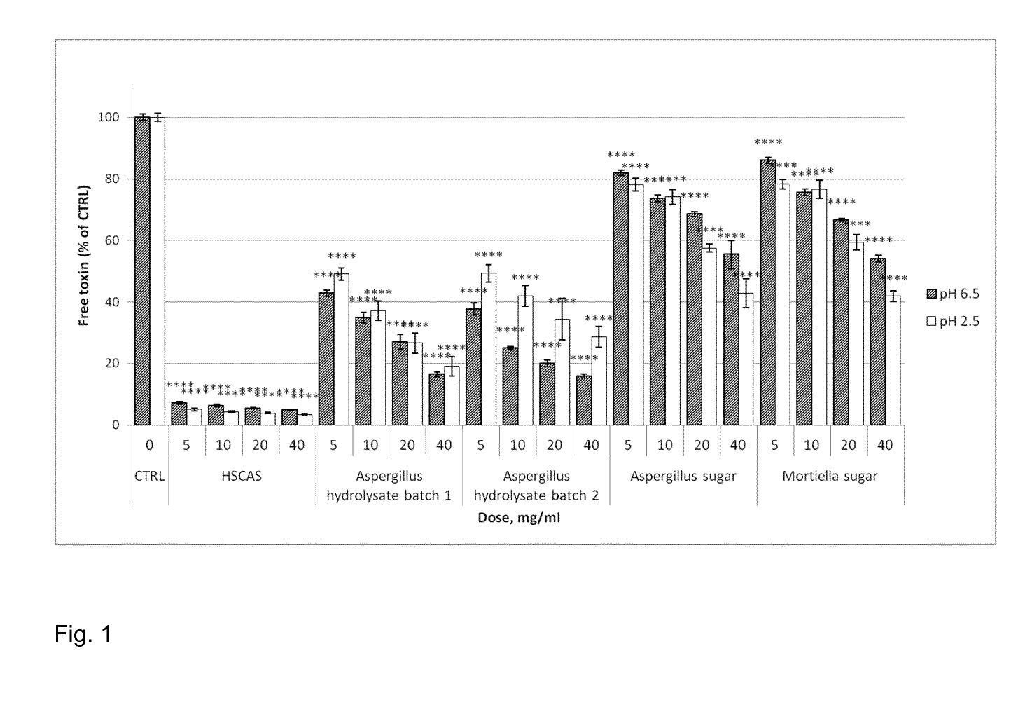

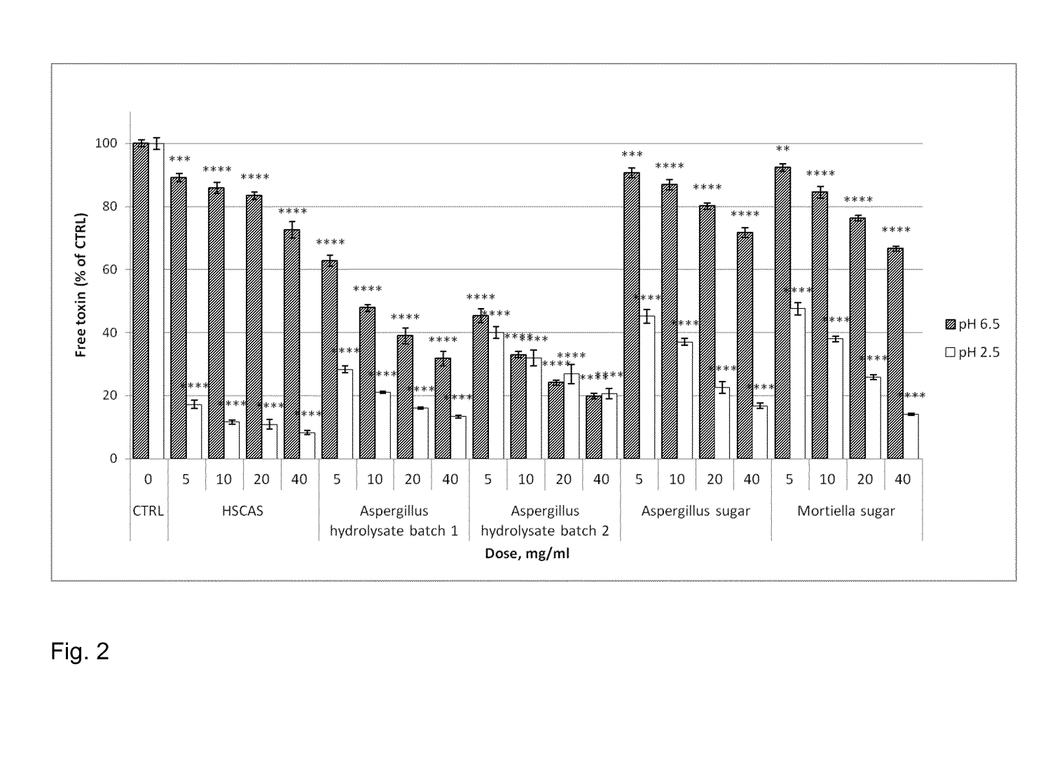

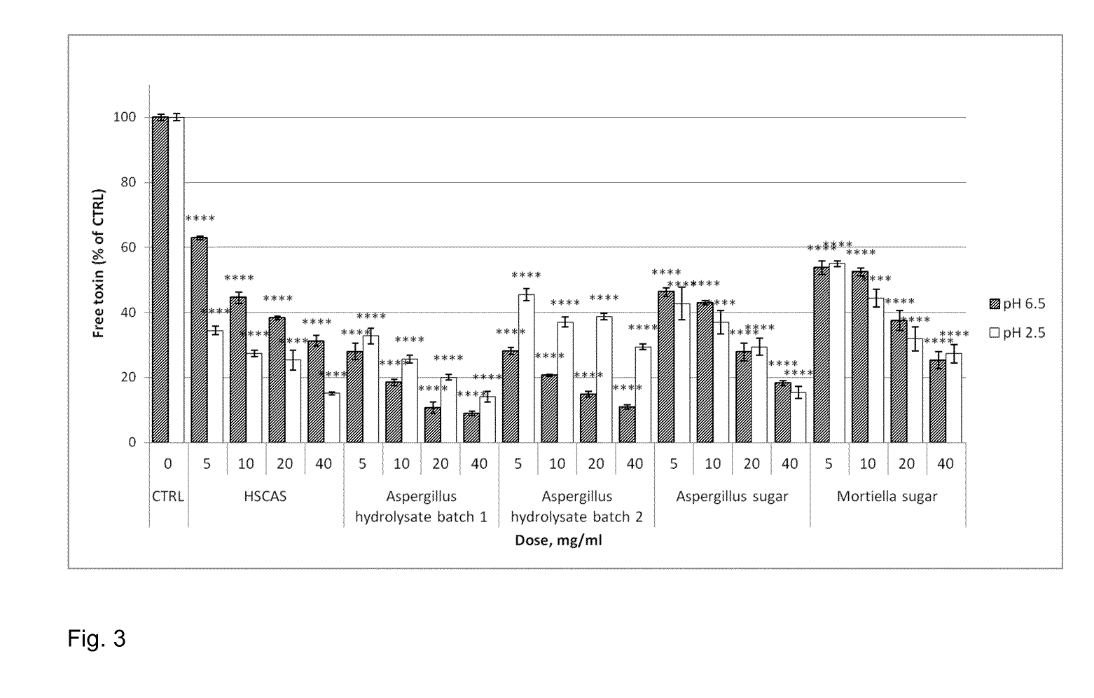

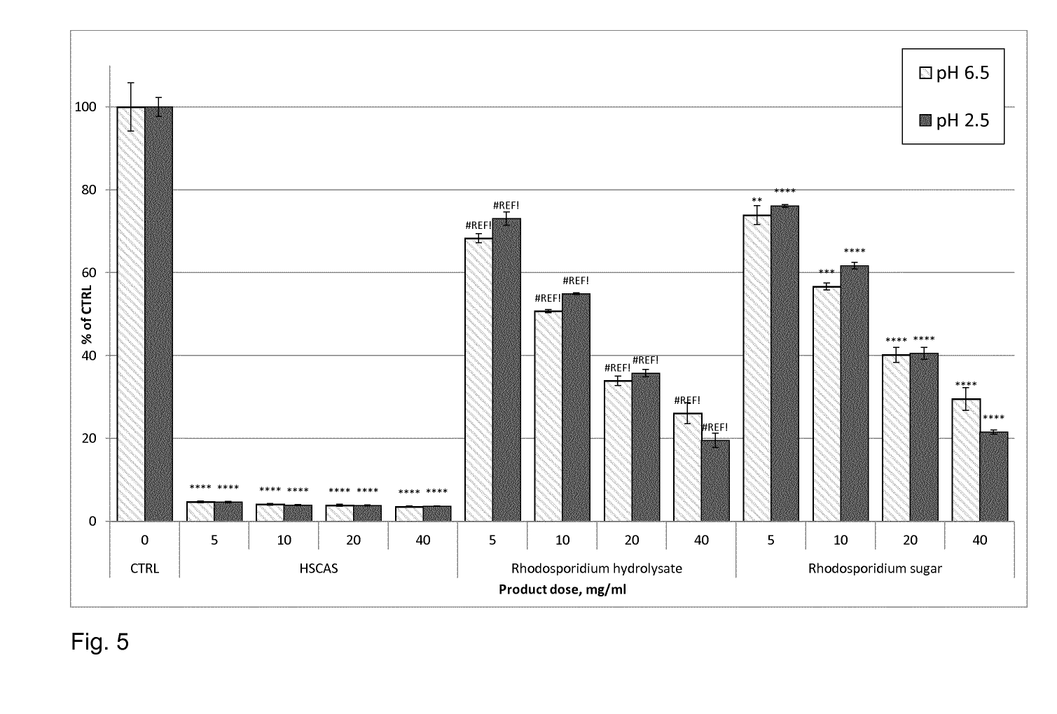

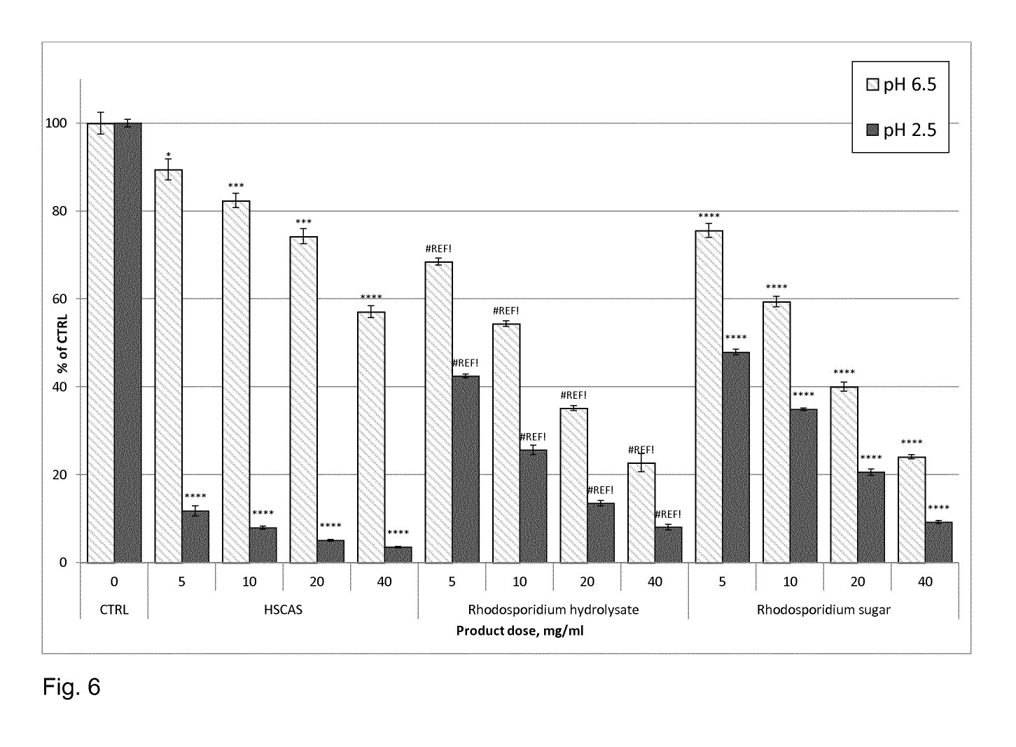

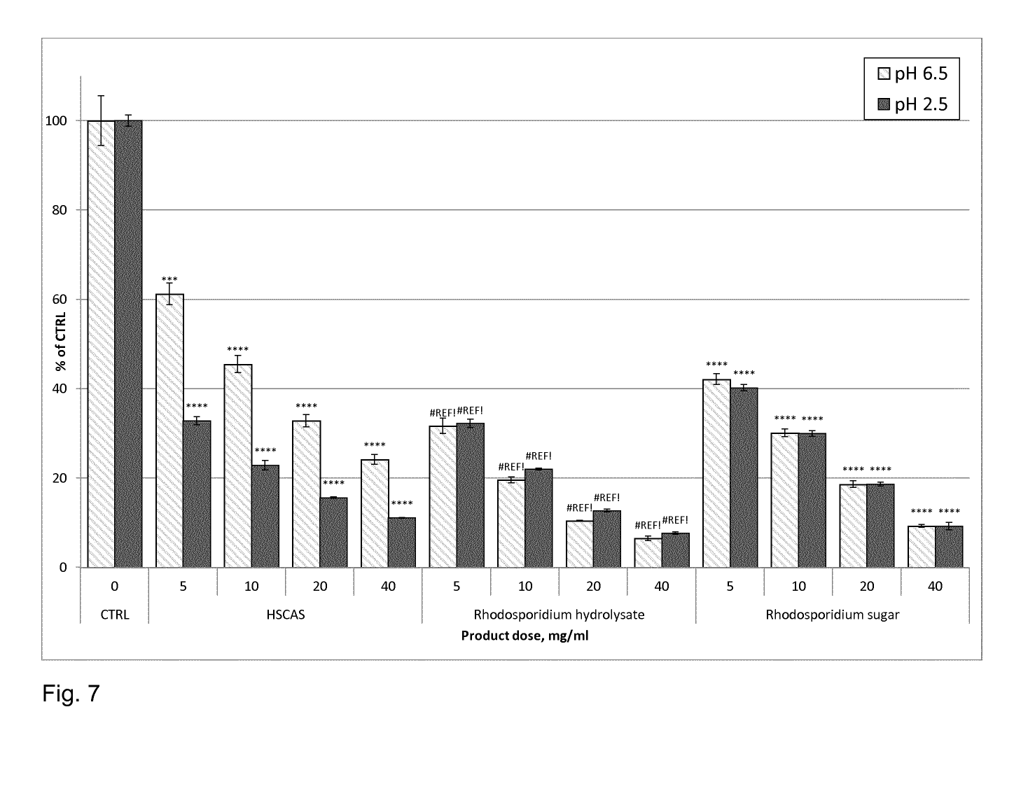

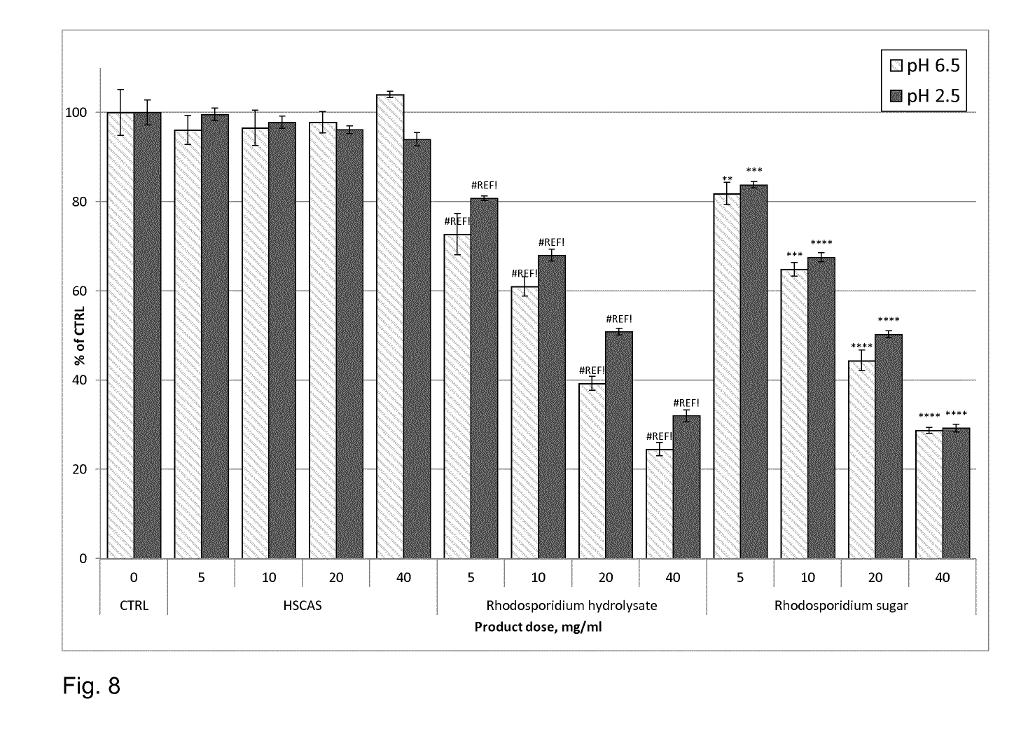

As shown in the experimental part of this disclosure, the test products bound all the tested mycotoxins: aflatoxin, ochratoxin, zearaleone, vomitoxin and in the case of yeast also Fumonisin B1 and T2 toxin. In contrast, the positive control hydrated sodium calcium aluminosilicate, HSCAS, was effective only in binding aflatoxin (FIGS. 1 and 5), ochratoxin at pH 2.5 (FIGS. 2 and 6), zearaleone A (FIGS. 3 and 7) and Fumonisin B1 (FIG. 8). HSCAS is a generic commercial product and an intensively studied adsorbing agent (Boudergue et al. 2009).

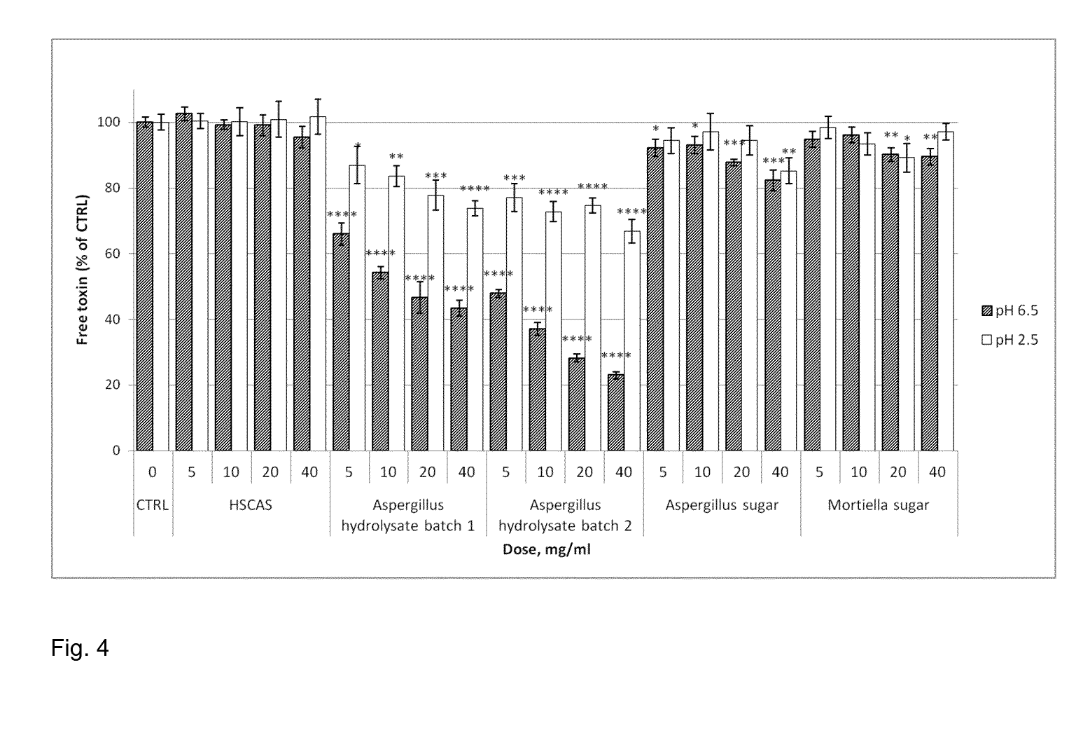

The microorganism biomass, in particular residual microorganism biomass has good binding properties both in the stomach (pH 2.5) and in small intestinal conditions (pH6.5), which is essential for a good mycotoxin adsorbing agent. Superior performance of the biomass grown on lignocellulose hydrolysate in binding vomitoxin (FIGS. 4 and 8) produces many specific exploitation options.

Significant differences in the mycotoxin binding capacity were observed between the test products. Aspergillus grown on lignocellulose hydrolysate (Aspergillus hydrolysate batches) showed consistent binding capability with all four mycotoxins. Binding was more effective at pH 6.5.

In conclusion, the microorganism biomass, in particular residual biomass grown on lignocellulose hydrolysate, binds a wider range of mycotoxins than the positive control hydrated sodium calcium aluminosilicate. It binds mycotoxins also at wider pH range than the positive control. Surprisingly, microorganism biomass, in particular the residual biomass grown on lignocellulose hydrolysate seems to be a more effective mycotoxin binder than the microorganism biomass, in particular residual biomass grown on pure saccharides.

The invention will hereafter be described by way of the following non-limiting items.

### Items

Item 1. A method for preparing a feed additive, said method comprising the steps of
(a) cultivating one or more microorganism(s) in a cultivation medium comprising lignocellulosic material,
(b) subject said microorganism(s) to a step of cell disruption to obtain non-living biomass of said microorganism(s)
(c) isolate a solid phase containing the microbial biomass and residues of lignocellulose of step (b),
(d) optionally, drying the microbial biomass obtained from step (c).

Item 2. The method of item 1, wherein the microbial biomass obtained from (c) is subjected to washing step.

Item 3. The method according to any of the preceding items, wherein said cultivation medium comprising lignocellulose hydrolysate.

Item 4. The method of according to any of the preceding items, wherein said cultivation medium comprises starch and/or sugar cane/beet derived sugars.

Item 5. The method of according to any of the preceding items, wherein the microbial biomass is obtained from oleaginous microorganisms.

Item 6. The method of according to any of the preceding items, wherein said microbial biomass is residual microbial biomass obtained from a fermentation process.

Item 7 The method of according to item 5, wherein the microbial biomass of step (c) is obtained by subjecting the oleaginous microorganisms to a step of removing the microbial oil from said microbial biomass.

Item 8. The method of according to item 7, wherein the microbial biomass of step (c) is obtained by subjecting the oleaginous microorganisms to a step of removing the microbial oil from said microbial biomass after step b).

Item 9. The method of according to any of the preceding items, wherein the microbial biomass is obtained from one or more microorganism.

Item 10. The method of according to any of the preceding items, wherein said microbial biomass is obtained from one or more fungi, preferably yeasts or filamentous fungi.

Item 11. The method of according to any of the preceding items, wherein said microbial biomass is obtained from Rhodosporidium.

Item 12. The method of according to any of the preceding items, wherein said microbial biomass is obtained from Aspergillus or Mortierella.

Item 13. A feed or food additive obtainable from the method of any one of items 1-12.

Item 14 A method for detoxifying a mycotoxin contaminated feed or food product, said method comprising the steps of
(a) providing a feed or food product contaminated with a mycotoxin,
(b) providing a feed or food additive according to item 13,
(c) mixing the feed or food product of (a) with the feed or food additive of (b).

Item 14. A method for reducing the bioavailability of mycotoxin in a mycotoxin contaminated feed or food product, said method comprising the steps of
(a) providing a feed or food product contaminated with a mycotoxin,
(b) providing a feed or food additive according to item 13,
(c) mixing the feed or food product of (a) with the feed or food additive of (b).

Item 15. A method for preparing a feed or food product, said method comprising the steps of
(a) providing a feed or food substance,
(b) providing a feed or food additive according to item 13,
(c) mixing the feed or food substance of (a) with the feed or food additive of (b) to obtain a feed or food product.

Item 16. The method according to any of items 14 to 15, wherein the feed additive amounts 0.001-20 wt % dry weight of the feed of step (c).

Item 17. The method according to any of items 14 to 15, wherein the feed additive amounts 0.1-3 wt % dry weight of the feed of step (c).

Item 18. The method according to any of items 14 to 17, wherein the feed substance is contaminated with one or more mycotoxin such as aflatoxin, ochratoxin, trichothecenes, vomitoxin, zearalenone, fumonisin or T2 toxin.

Item 19. A feed or food product obtainable from any one of items 15 to 18.

Item 20. A method for feeding an animal, said method comprising
(a) feeding said animal with a feed product according to item 19.

Item 21. The method according to item 20, wherein said animal is a ruminant or monogastric animal.

Item 22. The method according to item 20, wherein said animal is aquaculture.

Item 23. The method according to any of items 20 to 22, wherein the animal is a livestock.

Item 24. The method according to item 20, wherein said animal is a livestock selected from the list consisting of pigs, horses, poultry, cattle, goats and sheep.

Item 25. The method according to item 20, wherein said animal is a chicken.

Item 26. The method according to item 20, wherein said animal is a pig.

The invention is illustrated by the following non-limiting examples. The invention can be applicable to other mycotoxins than those illustrated in examples.

The invention can be applicable to biomasses from other microorganisms than those illustrated in examples. It should be understood, however, that the embodiments given in the description above and in the examples are for illustrative purposes only, and that various changes and modifications are possible within the scope of invention.

### EXAMPLES

### Example 1

### Preparation of Aspergillus Biomass Grown on Lignocellulose Hydrolysate

### Batch 1:

Hemicellulose hydrolysate was prepared from pelletized wheat straw by batch hot water extraction. Temperature of the extraction was 180° C. and the extraction time 1 hour. Solid material was removed from the hydrolysate by filtration. After this the hydrolysate was evaporated. The evaporated hydrolysate had 4.7 wt-% of phenolics in the whole dry matter content.

Filamentous fungus Aspergillus oryzae, strain DSM 1864 (or other A. oryzae strain, which are readily available from recognized microbial culture collections) was grown under aeration in a 5-liter fermentor. First 26 hours of the fermentation were done as a batch fermentation using sucrose. After this the fermentation was done as a continuous fermentation and hemicellulose medium (lignocellulose hydrolysate from which solid material had been removed as described above) was added continuously to the fermentor. Flow rate of the medium was 0.1 l/h and total cultivation time 98 h. Growth media was supplemented with yeast extract (5 g/l), (NH4)2SO4 (1.5 g/l), MgSO4 (1 g/l), KH2PO4 (1 g/l), K2HPO4 (2 g/l) and CaCl2 (0.1 g/l).

After cultivation biomass was inactivated by heat, harvested by filtration, washed using tap-water and freeze-dried. Dried biomass was pulverized by milling and oil extracted using n-heptane. Residual solvent was removed by drying the biomass by efficient ventilation.

The dried biomass was used in mycotoxin binding tests.

### Preparation of Aspergillus Biomass Grown on Lignocellulose Hydrolysate

### Batch 2:

Lignocellulose hydrolysate i.e. hemicellulose hydrolysate was prepared from wheat straw by thermo-chemical processing. After this the hydrolysate was evaporated. Before cultivation the evaporated hydrolysate was treated with activated charcoal to remove impurities and hydrolysed enzymatically to monomers.

After activated charcoal processing the hydrolysate had 3.4 w-% of phenolic compounds from the whole dry matter.

Filamentous fungus Aspergillus oryzae, strain DSM 1864 (or other A. oryzae strain which are readily available from recognized microbial culture collections) was grown under aeration in a 5-liter fermentor in fed-batch mode. Total fermentation time was 142 hours. Growth media (hemicellulose hydrolysate prepared as described above) was supplemented with yeast extract, (NH4)2SO4 (1.5 g/l), MgSO4 (1 g/l), KH2PO4 (1 g/l), K2HPO4 (2 g/l) and CaCl2 (0.1 g/l).

After cultivation biomass was inactivated by heat, harvested by filtration washed using tap-water and freeze-dried. Dried biomass was pulverized by milling and oil extracted using n-heptane. Residual solvent was removed by drying the biomass by efficient ventilation.

The dried biomass was used in mycotoxin binding tests.

### Preparation of Aspergillus and Mortierella Biomasses Grown on Pure Saccharides

In a similar manner Aspergillus oryzae strain DSM 1864 biomasses were grown on glucose in a 1200-I fermenter. The growth media was supplemented with Yeast Extract (10 g/L), (NH4)2SO4 (2.5 g/L), MgCl2×6H2O (1.78 g/L), K2HPO4 (1 g/L), KH2PO4 (2 g/L), CaCl2×2H2O (0.6 g/L), ZnSO4×7H2O (0.0003 g/L), CuCl×2H2O (0.0002 g/L), MnCl2×4H2O (0.0125 (g/L).

After cultivation biomasses were inactivated by heat, harvested by filtration, washed and dried. Dried biomass was mechanically disrupted by extrusion and oil extracted using n-hexane. The solvent was removed by heating the biomass to 50° C.

In a similar manner Mortierella isabeffina DSM 1414 strain (or other M. isabellina strain which are readily available from recognized microbial culture collections) biomasses were grown on glucose in a 1200-I fermenter. The growth media was supplemented with Yeast Extract (10 g/L), (NH4)2SO4 (2.5 g/L), MgCl2×6H2O (1.78 g/L), K2HPO4 (1 g/L), KH2PO4 (2 g/L), CaCl2×2H2O (0.6 g/L), ZnSO4×7H2O (0.0003 g/L), CuCl×2H2O (0.0002 g/L), MnCl2×4H2O (0.0125 (g/L)I).

After cultivation biomasses were inactivated by heat, harvested by filtration, washed and dried. Dried biomass was mechanically disrupted by extrusion and oil extracted using n-hexane. The solvent was removed by heating the biomass to 50° C.

These dried biomasses were used in mycotoxin binding tests.

### Preparation of Rhodosporidium Biomass Grown on Lignocellulose Hydrolysate

Lignocellulose hydrolysate was prepared from wheat straw by thermo-chemical and enzymatic processing. After this the hydrolysate was evaporated.

Rhodosporidium toruloides strain CBS 8587 (or other R. toruloides strain, which are readily available from recognized microbial culture collections) was grown under aeration in a 10-liter fermentor. Fermentation was done as fed-batch fermentation using lignocellulosic hydrolysate syrup as the carbon source. After 10 h batch fermentation lignocellulose hydrolysate syrup was added to the fermentor periodically during the 95 h cultivation. Growth medium was supplemented with yeast extract (8 g/l), (NH4)2SO4 (2.5 g/l), MgSO4 (2.5 g/l), KH2PO4 (3.5 g/l), K2HPO4 (1.5 g/l) and CaCl2 (0.1 g/l) and trace minerals ZnSO4 (0.0008 g/l), CuCl (0,00008 g/l), MnSO4 (0,0008 g/l), FeSO4 (0,0004 g/l) and NaH2PO4 (0.5 g/l).

After cultivation biomass was inactivated by heating, harvested by centrifugation washed using tap-water and dried in oven. Dried biomass was pulverized by milling and oil extracted using n-heptane. Residual solvent was removed by drying the biomass by efficient ventilation.

The dried biomass was used in mycotoxin binding tests.

### Preparation of Rhodosporidium Biomass Grown on Pure Saccharides

Rhodosporidium toruloides strain CBS 8587 (or other R. toruloides strain, which are readily available from recognized microbial culture collections) was grown under aeration in a pilot-scale fermentor. Fermentation was done as fed-batch fermentation using glucose as carbon source. After 24 h batch phase glucose syrup was added to the fermentor periodically during the 143 h cultivation. Growth medium was supplemented with yeast extract (8 g/l), (NH4)2SO4 (3 g/l), MgCl2 (2 g/l), K2HPO4 (9 g/l) and CaCl2 (0.4 g/l) and trace minerals ZnSO4 (0,0003 g/l), CuCl (0,0002 g/l) and MnCl2 (0.03 g/l).

After cultivation biomass was inactivated by heat, harvested by centrifugation, washed and dried. Dried biomass was pulverized by milling and oil extracted using n-heptane. The solvent was removed by heating the biomass to 50° C. The dried biomass was used in mycotoxin binding tests.

### Example 2

### Neutralisation of Mycotoxins by Binding

Test product suspensions were incubated in 50 mM phosphate buffer at pH 6.5 and 50 mM glycine-HCl at pH 2.5. After that they were incubated for two hours with tritium labeled mycotoxins (10 μg/l) by gently shaking at 37° C. The abundance of unbound mycotoxin was analysed from the supernatant. The test mycotoxins were Aflatoxin B1, Ochratoxin A, Zearaleone and Vomitoxin. In addition, Fumonisin B1 and T2 toxin were used in the tests with Rhodosporidium yeast biomasses.

The test products were: Aspergillus residual biomass grown on lignocellulose hydrolysate (in FIGS. 1, 2, 3 and 4 Aspergillus hydrolysate batch 1 and Aspergillus hydrolysate batch 2), Aspergillus residual biomass grown on pure saccharides, Mortierella biomass grown on pure saccharides. (in FIGS. 1, 2, 3, and 4 Aspergillus sugar and Mortierella sugar), Rhodosporidium residual biomass grown on lignocellulose hydrolysate (In FIGS. 5, 6, 7, 8, 9 and 10 Rhodosporidium hydrolysate) and Rhodosporidium residual biomass grown on pure saccharides (In FIGS. 5, 6, 7, 8, 9 and 10 Rhodosporidium sugar).

The mycotoxin binding experiment was carried out in four replicate and in 4 doses: 5, 10, 20 and 40 mg/ml.

As positive controls were used HSCAS in 4 dose levels.

As negative control was used no amendment.

### Test Treatments

### Aflatoxin Binding by Fungal and Yeast Biomasses.

As shown in FIGS. 1 and 5 the concentration of free aflatoxin in the presence of potential binders as compared to control with no binder at pH 6.5 (columns with dark stripe) and pH 2.5 (white columns). Error bars indicate standard error (SE) between 4 replicate reaction vessels and asterisks the statistical significance of the difference to control according to the Student's t-test (p-value<0.05*, p-value<0.01 **, p-value<0.001 ***, p-value<0.0001 ****). As shown in FIGS. 1 and 5, all test products showed some binding of aflatoxin (AFB1).

### Ochratoxin Binding by Fungal and Yeast Biomasses.

As is shown in FIGS. 2 and 6 the concentration of free ochratoxin in the presence of potential binders as compared to control with no binder at pH 6.5 (columns with dark stripe) and pH 2.5 (white columns). Error bars indicate SE between 4 replicate reaction vessels and asterisks the statistical significance of the difference to control according to the Student's t-test (p-value<0.05 *, p-value<0.01 **, p-value<0.001 ***, p-value<0.0001 ****). As shown in FIGS. 2 and 6, Aspergillus hydrolysates bind the toxin also at pH 6.5, whereas the control HCAS binds ochratoxin only at low pH.

### Zearalenone Binding by Fungal and Yeast Biomasses.

As is shown in FIGS. 3 and 7 the concentration of free zearalenone in the presence of potential binders as compared to control with no binder at pH 6.5 (columns with dark stripe) and pH 2.5 (white columns). Error bars indicate SE between 4 replicate reaction vessels and asterisks the statistical significance of the difference to control according to the Student's t-test (p-value<0.05 *, p-value<0.01 **, p-value<0.001 ***, p-value<0.0001 ****). As shown in FIGS. 3 and 7 all the test products were binding ZEA. In addition the text products showed efficient binding of zearaleone even at pH 6.5.

### Vomitoxin Binding by Fungal and Yeast Biomasses.

As is shown in FIGS. 4 and 8 the concentration of free vomitoxin in the presence of potential binders as compared to control with no binder at pH 6.5 (columns with dark stripe) and pH 2.5 (white columns). Error bars indicate SE between 4 replicate reaction vessels and asterisks the statistical significance of the difference to control according to the Student's t-test (p-value<0.05 *, p-value<0.01 **, p-value<0.001 ***, p-value<0.0001 ****). As shown in FIG. 4, all the test products were binding vomitoxin efficiently at pH 6.5. As shown in FIG. 8, the yeast test products were binding vomitoxin efficiently in both tested pH conditions. The control did not bind vomitoxin.

### Fumonisin B1 Binding by Yeast Biomass

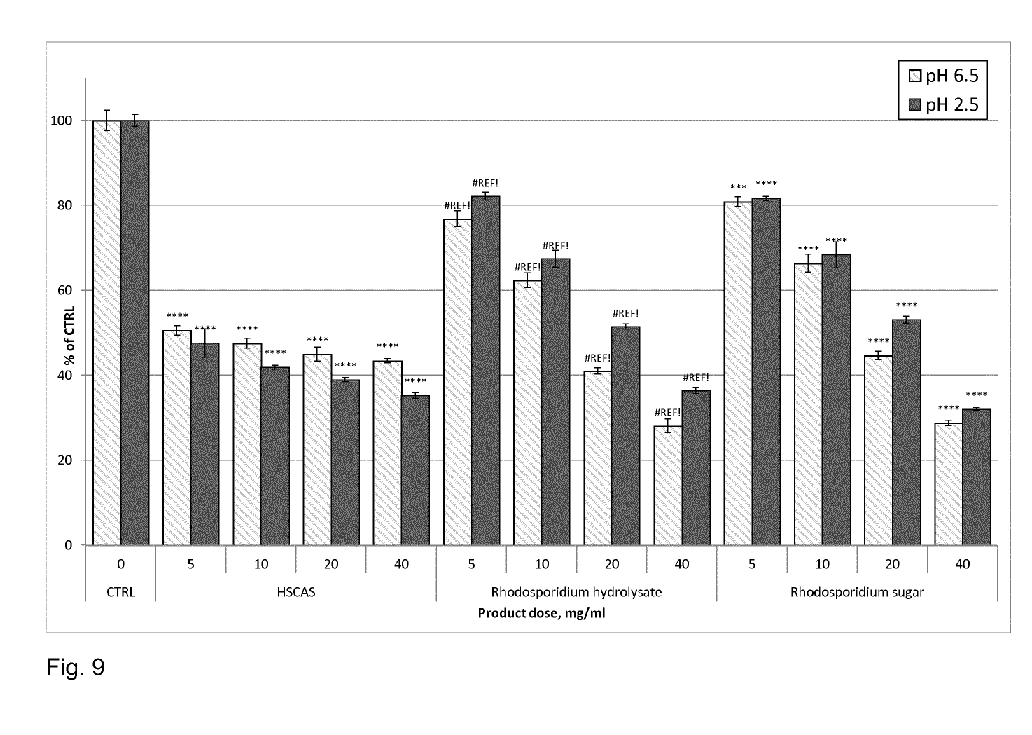

As is shown in FIG. 9 the concentration of free Fumonisin B1 in the presence of potential binders as compared to control with no binder at pH 6.5 (columns with dark stripe) and pH 2.5 (white columns). Error bars indicate SE between 4 replicate reaction vessels and asterisks the statistical significance of the difference to control according to the Student's t-test (p-value<0.05 *, p-value<0.01 **, p-value<0.001 ***, p-value<0.0001 ****). A shown in FIG. 9, both test products showed some binding of Fumonisin B1.

### T2 Toxin Binding by Yeast Biomass

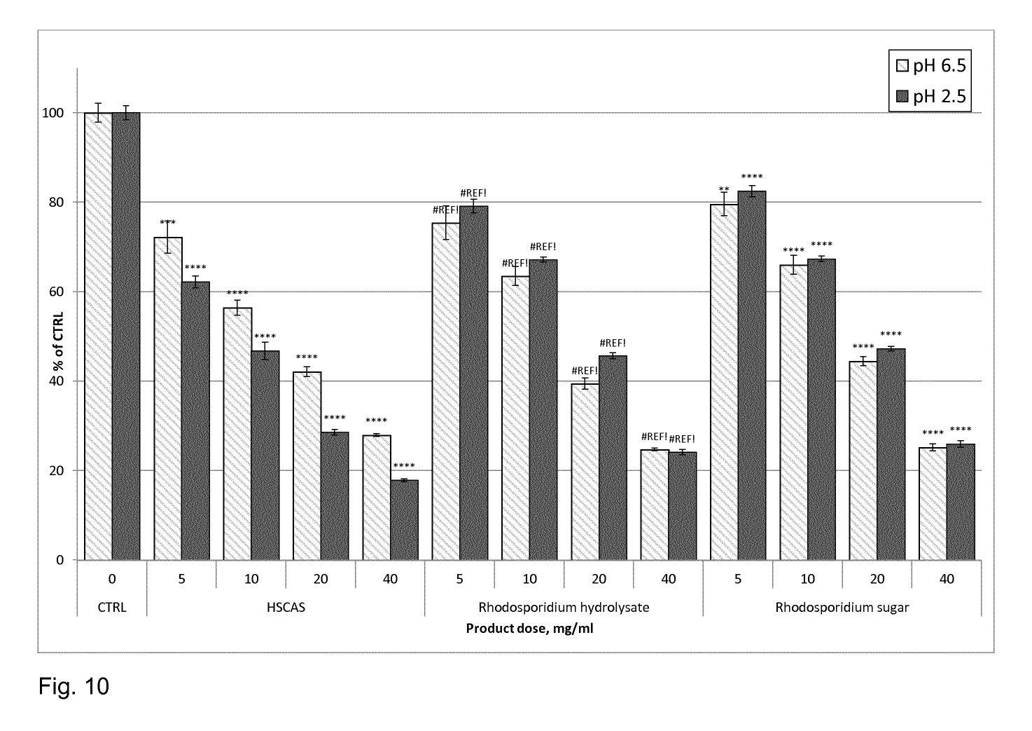

As is shown in FIG. 10 the concentration of free T2 toxin in the presence of potential binders as compared to control with no binder at pH 6.5 (columns with dark stripe) and pH 2.5 (white columns). Error bars indicate SE between 4 replicate reaction vessels and asterisks the statistical significance of the difference to control according to the Student's t-test (p-value<0.05 *, p-value<0.01 **, p-value<0.001 ***, p-value<0.0001 ****). A shown in FIG. 10, both test products showed some binding of T2 toxin.

The aflatoxin binding capacity of the test products was independent of pH. The Aspergillus biomasses grown on lignocellulose hydrolysate were comparable to HSCAS. At low pH ochratoxin binding was equal (stomach), but at pH 6.5 the test products outperformed the control. This was noticeable in small intestine and especially with Aspergillus and Rhodosporidium biomasses grown on lignocellulose hydrolysate. Zearaleone binding at pH 6.5 was more effective with Aspergillus biomass grown on lignocellulose hydrolysates than with the control. Only Aspergillus and Rhodosporidium biomasses grown on lignocellulose hydrolysates were able to bind vomitoxin. The binding was more effective at neutral than at acidic pH.

The results indicate that microbial biomasses obtained from cultivation with lignocellulose hydrolysates were able to bind mycotoxins more efficiently than microbial biomasses obtained from cultivation with pure saccharides.

1. Microorganism biomass for use in preventing or reducing the adverse effects of mycotoxins in animal or human digestive tract, characterized in that said biomass comprises non-living microorganism biomass obtainable by cultivating microorganism strains on a cultivation medium comprising lignocellulosic material.

2. The biomass for use in preventing or reducing the adverse effects of mycotoxins in animal or human digestive tract according to claim 1, characterized in that the cultivation medium comprises lignocellulose hydrolysate.

3. The biomass for use in preventing or reducing the adverse effects of mycotoxins in animal or human digestive tract according to claim 1, characterized in that the microorganism biomass comprises residues of lignocellulose.

4. The biomass for use in preventing or reducing the adverse effects of mycotoxins in animal or human digestive tract according to claim 1, characterized in that the microorganism biomass is residual microorganism biomass from a single cell oil production process.

5. The biomass for use in preventing or reducing the adverse effects of mycotoxins in animal or human digestive tract according to claim 1, characterized in that the microorganism biomass is obtainable by cultivating fungi strains, preferably yeasts or filamentous fungi.

6. The biomass for use in preventing or reducing the adverse effects of mycotoxins in animal or human digestive tract according to claim 1, characterized in that the microorganism biomass is obtainable by cultivating filamentous fungi strains, preferably strains of Aspergillus and/or Mortierella genus or yeasts, preferably strains of Rhodosporidium or Lipomyces genus.

7. The biomass for use in preventing or reducing the adverse effects of mycotoxins in animal or human digestive tract according to claim 1, characterized in the microorganism biomass prevents or reduces the adverse effects of mycotoxins in animal or human stomach, small intestine and/or colon.

8. The biomass for use in preventing or reducing the adverse effects of mycotoxins in animal or human digestive tract according to claim 1, characterized in the microorganism biomass prevents or reduces the adverse effects of mycotoxins aflatoxin, ochratoxin, trichothecenes, vomitoxin, zearalenone, fumonisin and/T2 toxin.

9. A food or feed composition for use in preventing or reducing the adverse effects of mycotoxins in animal or human digestive tract, characterized in that it comprises the microorganism biomass according to claim 1.

10. The composition for use in preventing or reducing the adverse effects of mycotoxins in animal or human digestive tract according to claim 9, characterized in that the composition comprises the microorganism biomass 0.001-20 wt %, preferably 0.1-3 wt % dry weight of dry weight of said composition.

11. The composition for use in preventing or reducing the adverse effects of mycotoxins in animal or human digestive tract according to claim 9, characterized in that the composition is a ruminant or monogastric animal feed composition.

12. The composition for use in preventing or reducing the adverse effects of mycotoxins in human or animal digestive tract according to claim 9, characterized in that the food composition is a nutritional product for human use.

13. The composition for use in preventing or reducing the adverse effects of mycotoxins in animal or human digestive tract according to claim 9, characterized in that said microorganism biomass is used in the composition as a food or feed topping, food or feed extender or food or feed supplement.

14. A feed or food composition, characterized in that the composition comprises 0.001-20 wt % dry weight of dry weight of said composition non-living microorganism biomass as defined in claim 1.

15. The composition according to claim 14, characterized in that the composition comprises 0.1-3 wt % dry weight of dry weight of said composition microorganism biomass.

16. A method for improving the well-being and increasing the productivity of an animal, characterized in that said method comprises feeding to said animal a feed composition comprising non-living microorganism biomass as defined in claim 1.

17. The method according to claim 16, characterized in that the composition comprises 0.001-20 wt %, preferably 0.1-3 wt % dry weight of dry weight of said composition microorganism biomass.

18. A method for preparing a feed additive, said method comprising the steps of
(a) cultivating one or more microorganism(s) in a cultivation medium comprising lignocellulosic material,
(b) subject said microorganism(s) to a step of cell disruption to obtain non-living biomass of said microorganism(s)
(c) isolate a solid phase containing the microbial biomass and residues of lignocellulose of step (b),
(d) optionally, drying the microbial biomass obtained from step (c).

19. The method of claim 18, wherein the microbial biomass obtained from (c) is subjected to washing step.

20. The method according to claim 18, wherein said cultivation medium comprising lignocellulose hydrolysate.

21. The method of according to claim 18, wherein said cultivation medium comprises starch and/or sugar cane/beet derived sugars.

22. The method of according to any of the preceding claim 18, wherein the microbial biomass is obtained from oleaginous microorganisms.

23. The method of according to claim 18, wherein said microbial biomass is residual microbial biomass obtained from a fermentation process.

24. The method of according to claim 22, wherein the microbial biomass of step (c) is obtained by subjecting the oleaginous microorganisms to a step of removing the microbial oil from said microbial biomass.

25. The method of according to claim 24, wherein the microbial biomass of step (c) is obtained by subjecting the oleaginous microorganisms to a step of removing the microbial oil from said microbial biomass after step b).

26. The method of according to claim 18, wherein the microbial biomass is obtained from one or more microorganism.

27. The method of according to claim 18, wherein said microbial biomass is obtained from one or more fungi, preferably yeasts or filamentous fungi.

28. The method of according to claim 18, wherein said microbial biomass is obtained from Rhodosporidium.

29. The method of according to claim 18, wherein said microbial biomass is obtained from Aspergillus or Mortierella.

30. A feed or food additive obtainable from the method of claim 18.

31. A method for detoxifying a mycotoxin contaminated feed or food product, said method comprising the steps of
(a) providing a feed or food product contaminated with a mycotoxin,
(b) providing a feed or food additive according to claim 30,
(c) mixing the feed or food product of (a) with the feed or food additive of (b).

32. A method for reducing the bioavailability of mycotoxin in a mycotoxin contaminated feed or food product, said method comprising the steps of
(a) providing a feed or food product contaminated with a mycotoxin,
(b) providing a feed or food additive according to claim 30,
(c) mixing the feed or food product of (a) with the feed or food additive of (b).

33. A method for preparing a feed or food product, said method comprising the steps of
(a) providing a feed or food substance,
(b) providing a feed or food additive according to claim 30,
(c) mixing the feed or food substance of (a) with the feed or food additive of (b) to obtain a feed or food product.

34. The method according to claim 31, wherein the feed additive amounts 0.001-20 wt % dry weight of the feed of step (c).

35. The method according to claim 31, wherein the feed additive amounts 0.1-3 wt % dry weight of the feed of step (c).

36. The method according to claim 31, wherein the feed substance is contaminated with one or more mycotoxin such as aflatoxin, ochratoxin, trichothecenes, vomitoxin, zearalenone, fumonisin or T2 toxin.

37. A feed or food product obtainable from claim 33.

38. A method for feeding an animal, said method comprising
(a) feeding said animal with a feed product according to claim 37.

39. The method according to claim 38, wherein said animal is a ruminant or monogastric animal.

40. The method according to claim 38, wherein said animal is aquaculture.

41. The method according to claim 38, wherein the animal is a livestock.

42. The method according to claim 38, wherein said animal is a livestock selected from the list consisting of pigs, horses, poultry, cattle, goats and sheep.

43. The method according to claim 38, wherein said animal is a chicken.

44. The method according to claim 38, wherein said animal is a pig.
